(19) United States
(12) Patent Application Publication
(10) Pub. No.: US 2008/0208348 A1
(43) Pub. Date: Aug. 28, 2008

(54) Apparatus and Method for Shoulder Arthroplasty

(76) Inventor: Wolfgang Fitz, Natick, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/816,132

(22) PCT Filed: Feb. 10, 2006
(86) PCT No.: PCT/US2006/004673

§ 371 Date: Aug. 14, 2007

Related U.S. Application Data
(60) Provisional application No. 60/652,031, filed on Feb. 11, 2005.

(51) Int. Cl. A61F 2/40

(52) U.S. Cl. 623/19.14; 623/19.11; 623/19.13; 623/19.12

(57) ABSTRACT

A glenoid prosthesis can include a bearing shell for accommodating the head of the humerus and a stem extending at an angle from a reverse face of the shell so as to engage the stem within a canal in the lateral border of the scapula for anchoring the glenoid prosthesis to the scapula. The prosthesis can be one piece or modular, where the shell and stem can be chosen from a plurality of shapes and sizes to most closely match the natural anatomy of the joint. A fastening means can attach the shell to the stem and a wearing surface can be secured to the face of the shell. The stem can be sized slightly larger than the canal and forcibly inserted into the canal to anchor the glenoid prosthesis to the scapula. Flutes, barbs, threads, etc. on the stem can further secure the stem within the canal.

### RELATED APPLICATIONS

This application claims priority to, and incorporates by reference, the entire disclosure of U.S. Provisional Patent Application No. 60/652,031, filed on Feb. 11, 2005.

### FIELD

The invention relates to a glenoid component for a shoulder prosthesis. More particularly, the invention relates to a glenoid component having a stem extending at an angle from a reverse face of the glenoid prosthesis so as to engage the stem within a canal in the lateral border of the scapula for anchoring the glenoid prosthesis to the scapula.

### BACKGROUND

Prosthetic shoulder joints are well known in the art and include many different types and shapes of glenoid and humeral components, which form the two major components of the human shoulder joint. The upper end of the upper arm or humerus forms a ball-like structure, the humeral component, which fits into a socket-like structure of the scapula, the glenoid component. A typical glenoid component includes a generally circular body having a concave surface and an opposing medial surface. The scapula is resected to remove the natural glenoid and one or more cavities are formed in the neck of the scapula. One or more protrusions on the medial surface of the glenoid component can mate with the cavities to secure the glenoid component to the scapula.

Despite the numerous advances in the designs of glenoid components and the methods and tools used for their installation, these prostheses still lack the stability and strength of natural healthy glenoid components and the relative positions of the prosthetic glenoid and humeral components most often does not provide the proper soft tissue balance.

Thus, long-term results of total shoulder replacement are still unsatisfactory when compared to recent advances in hip and knee replacement. This can be traced to glenoid component loosening and prosthesis malpositioning with excessive anteversion or retroversion of the glenoid components. For a successful outcome, the biometrics of the shoulder have to be reconstructed by careful restoration of muscle tension as well as glenoid retroversion. In addition, the superoinferior tilt of the glenoid is important in its role in eccentric component loading.

However, a precise restoration of glenoid orientation is often complicated by the bone destruction, which resulted in the need for the shoulder replacement. Unlike hip or knee replacements, which rely heavily on the intramedullary canal for fixation and alignment, no comparable anatomical structure in the shoulder has been described. Therefore, evaluation of the anteroposterior and superoinferior tilt of the glenoid component is often based on palpation and experience.

### SUMMARY

Provided herein are apparatus and methods and compositions for shoulder arthroplasty. A glenoid prosthesis can include a bearing shell for accommodating the head (or prosthetic ball) of the humerus on a front face and a stem extending at an angle from a reverse face of the shell to engage the stem within a canal in the lateral border of the scapula for anchoring the glenoid prosthesis to the scapula.

The shell and stem can be one-piece or modular. In a modular prosthesis, a combination of a shell and stem can be chosen from a number of various shapes and sizes of shells and stems so as to most closely match the natural anatomy of the joint undergoing arthroplasty, and a fastening means can attach the shell to the stem. A wearing surface also can be secured to the face of the shell. The stem can be sized slightly larger than the canal and forcibly inserted into the canal to anchor the glenoid prosthesis to the scapula. The stem can include flutes, barbs, threads, etc. to further secure the stem within the canal. One prosthesis can have a stem in the form of a screw, which aligns with and is threaded into the canal. The screw can be further used to secure the bearing shell to the stem. Additional pins, pegs, screws, etc. can be used in securing the glenoid prosthesis to the scapula.

In one embodiment, a glenoid prosthesis for use in shoulder arthroplasty can include a shell component having a first face configured to contact a surface of the scapula and a stem configured to extend from the first face into a canal in a lateral border of the scapula and configured to engage within the canal for anchoring the glenoid prosthesis to the scapula. The shell can have an opening therethrough such that the stem can be inserted into the canal through the opening. The glenoid prosthesis further can include an attachment means for securing the shell to the stem.

In various aspects, the stem can be tapered to conform to a narrowing of the canal. The opening can be tapered to conform to the stem with a dimension of the opening at the first face being smaller than a corresponding dimension of the end of the stem, such that the stem can be wedged within the opening. The end of the stem can be threaded to mate with matching threads in the opening of the shell to secure the shell to the stem. The end of the stem can be flanged to engage a shoulder formed within the opening. As the stem is further engaged into the canal, a force is exerted by the flange against the shoulder to secure the shell to the stem. A bolt can be threaded on the end of the stem, such that the head of the bolt can engage the shoulder as the bolt is threaded.

In various aspects, the stem can be threaded, fluted, and/or can have barbs extending outwardly from the outer surface for engaging the stem within the canal. The stem can have a longitudinal opening extending at least partway through the stem with a number of bores extending from an outer surface of the stem to intersect the longitudinal opening. Adhesive can be inserted into the longitudinal opening and through the bores to at least partially fill the canal. The outer surface of the stem can be porous or can include a plurality of depressions and/or for engaging with an adhesive within the canal.

Generally, a length and diameter of the stem can correspond to a length and diameter of the canal. Further, the angle formed between the stem and the shell can correspond to the angle between the canal and the natural glenoid of the shoulder.

In one embodiment, a method of performing shoulder arthroplasty can include preparing the glenoid region of the scapula so as to obtain access to a canal in a lateral border of the scapula, further preparing the glenoid region to accommodate a shell component of a glenoid prosthesis configured for articulation of a natural or prosthetic humeral head therein, and providing a stem configured to extend from the glenoid region into the canal and configured to engage within the canal for anchoring the glenoid prosthesis to the scapula.

### BRIEF DESCRIPTION OF THE DRAWINGS

FIG. 1 illustrates an anterior view of the shoulder prosthesis components when assembled and located relative to the scapula and humerus.

FIG. 2 illustrates a lateral view of the shoulder prosthesis components when assembled and located relative to the scapula and humerus.

FIG. 3 illustrates a cross-sectional view of the glenoid component and scapular stem of the shoulder prosthesis of FIGS. 1 and 2, taken along line 3-3 of FIG. 2.

FIGS. 4A-4C illustrate cross-sectional views corresponding to that of FIG. 3 of various embodiments of the glenoid component and scapular stem assembly.

FIGS. 5A-5D illustrate transverse cross-sectional views of various embodiments of the scapular stem of the shoulder prosthesis of FIGS. 1 and 2, taken along line 5-5 of FIGS. 1 and 2.

### DETAILED DESCRIPTION

Referring to FIGS. I and 2, a shoulder prosthesis 10 is illustrated. The shoulder prosthesis 10 can be used in total shoulder arthroplasty and can include a humerus prosthesis 12 and a glenoid prosthesis or assembly 14. The glenoid assembly 14 can include a glenoid component 16 and a scapular stem 18. The humerus prosthesis 12 can include anchorage means, such as humeral anchor 20, and head 22 that can mate with the glenoid assembly 14. Other anchorage means for humerus prosthesis 12 can be contemplated, including pins, pegs, screws, adhesives and/or other known means for anchoring a humeral prosthesis to the humerus H. Preferably, humerus prosthesis 12 can have the general shape of the natural humerus and head of the shoulder, though other shapes that mate with glenoid assembly 14, as will be further described, may be contemplated. When replacement of the humeral head is not indicated, the glenoid assembly 14 can be used in the arthroplasty procedure by itself, with the glenoid component 16 fabricated to mate with the natural head of the humerus H.

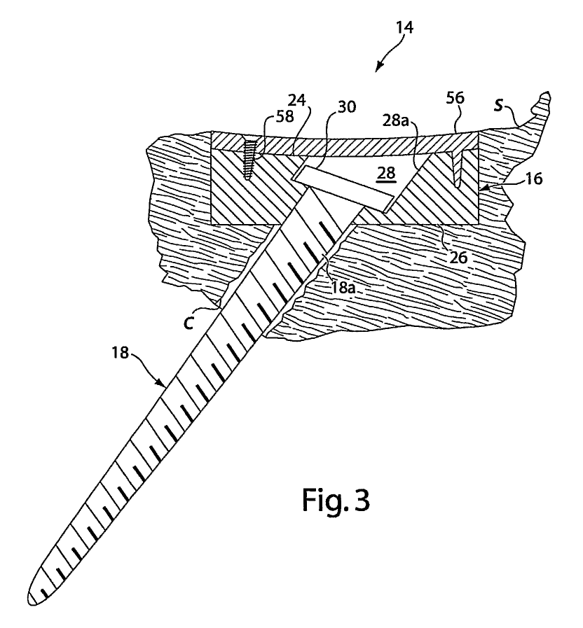

Referring also to FIG. 3, the glenoid assembly 14 can be described in more detail. The glenoid assembly or prosthesis 14 can include a glenoid component, or bearing shell 16 for accommodating the head (or prosthetic ball) of the humerus on a front face 24 of the shell 16. The scapular stem 18 can have a generally frustoconical shape and can extend at an angle from a reverse face 26 of the shell 16 to engage the stem 18 within a canal C in the lateral border of the scapula S for anchoring the glenoid prosthesis 14 to the scapula S.

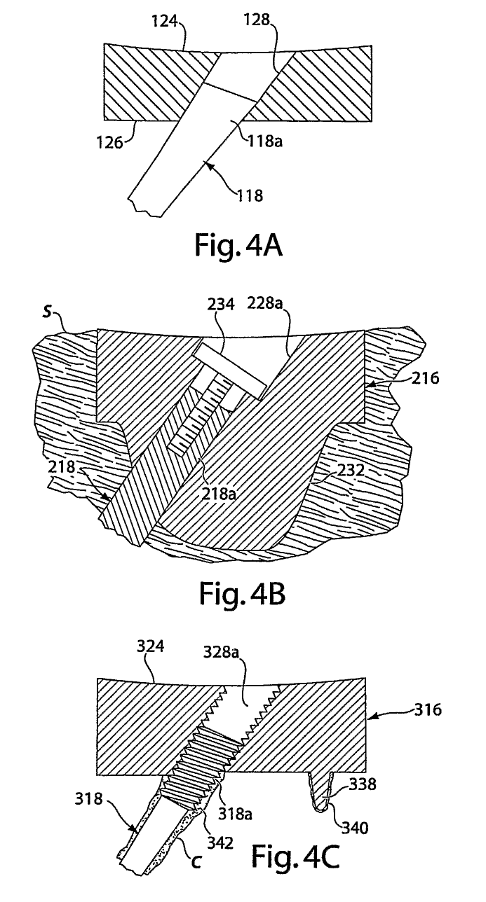

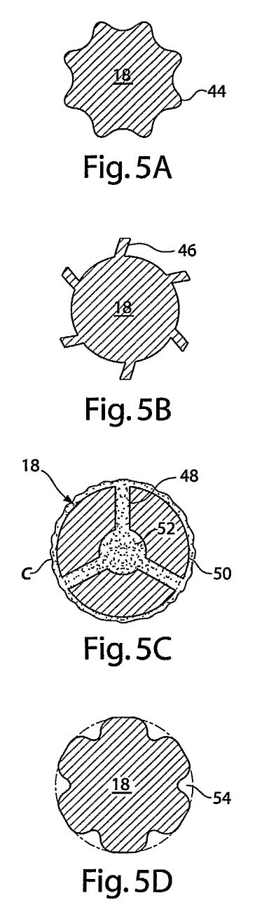

The shell 16 and stem 18 can be one-piece or modular. In a modular embodiment, as indicated in FIG. 3, a combination of a shell 16 and stem 18 can be chosen from a plurality of shapes and sizes of shells and stems so as to most closely match the natural anatomy of the joint undergoing arthroplasty. For the embodiment of FIG. 3, shell 16 can be positioned on the scapula S to closely match the orientation of the natural glenoid. Shell 16 can have an opening 28 therethrough, which can be generally aligned with canal C when shell 16 is so positioned on scapula S. The stem 18 can be inserted through opening 28 and into canal C. The stem 18 can be in the form of a screw, which aligns with and is threaded into the canal C. The end 18a of stem 18 proximate shell 16 can include flange 30, which can to bear against a countersunk portion 28a of opening 28 as stem 18 is threaded and tightened into canal C, thus securing shell 16 to scapula S. As further described herein with respect to FIGS. 4A-4C, shell 16 can be at least partially secured to scapula S prior to inserting stem 18 through opening 28. Other configurations of stem 18 can be contemplated, as further described herein with respect to FIGS. 5A-5D.

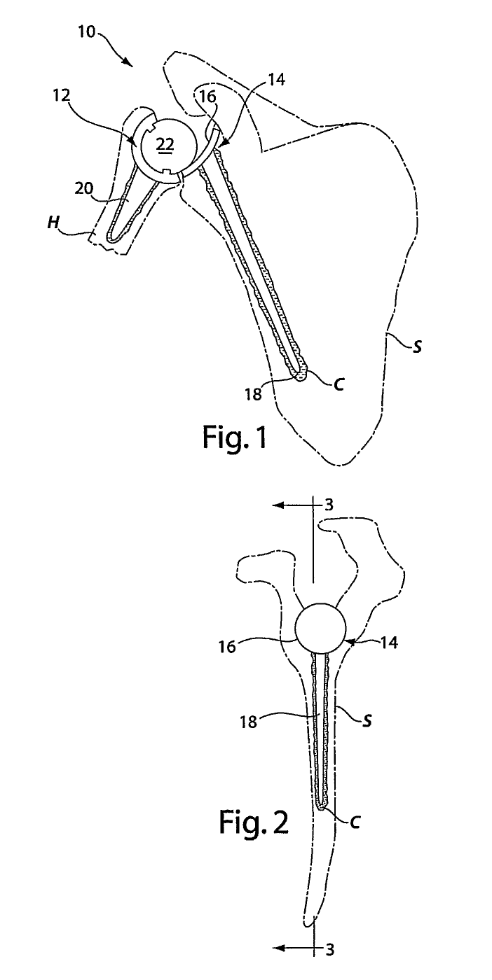

Referring now to FIGS. 4A-4C, various embodiments of glenoid assembly 14 are illustrated in longitudinal cross-section, wherein components have reference numbers offset by multiples of one hundred to those of corresponding components in FIGS. 1-3. For the embodiment of FIG. 4A, stem 118 and opening 128 can have matching tapers, with opening 128 on front face 124 being larger than end 118a of stem 118 and opening 128 on reverse face 126 being smaller than end 118a. Thus, stem 118 can be inserted through opening 128 into canal C until the taper of stem 118 engages the taper of opening 128, with end 118a partway through opening 128. As stem 118 is driven further into canal C, the tapered engagement can secure shell 116 to scapula S.

For the embodiment of FIG. 4B, shell 216 can include a keel 232 that can secure shell 216 to scapula S, as is known in the art. Shell 216 and keel 232 can include opening 228 for insertion of stem 218 therethrough and into canal C. For the embodiment of FIG. 4B, end 218a of stem 218 can be internally threaded to receive matching threaded bolt 234 therein. When stem 218 is fully inserted into canal C, end 218a can be within opening 228, but not extending into a countersunk portion 228a of opening 228. Head 236 of bolt 234 can bear against countersunk portion 228a of opening 228 as bolt 234 is threaded into end 218a, thus securing shell 216 to scapula S. Alternately, it can be understood that end 218a can include external threads instead of internal threads and bolt 234 can be configured as an internally threaded sleeve.

For the embodiment of FIG. 4C, shell 316 can include additional attachment means for securing shell 316 to scapula S. As illustrated in FIG. 4C, pin 338 can be press fit into a previously prepared recess 340 in scapula S. Other attachment means for shell 316 can be contemplated, including pegs, screws, adhesives and/or other known means for anchoring a glenoid prosthesis to scapula S. For the embodiment of FIG. 4C, adhesive 342 can be placed in canal C prior to insertion of stem 318 through opening 328. Opening 328 can be internally threaded and end 318a of stem 318 can include matching threads. Stem 318 is inserted through opening 328 into canal C and end 318a is threaded into opening 328 until end 318a is below front face 324 of shell 316. When cured, adhesive 342 can secure stem 318 in canal C.

As described herein, FIG. 3 illustrates an embodiment of glenoid prosthesis 14 having a screw configuration of stem 18. FIGS. 5A-5D can illustrate various alternate embodiments of stem 18 in transverse cross-section. FIG. 5A illustrates a fluted cross-section with eight flutes 44. Flutes 44 can be sized to engage the walls of canal C as stem 18 is inserted therein and other numbers of flutes can be provided. FIG. 5B illustrates stem 18 having a generally circular cross-section with a plurality of barbs 46 thereon for engaging the walls of canal C. For the embodiments of FIGS. 5A and 5B, stem 18 can be sized slightly larger than the canal C and forcibly inserted into the canal C to anchor the glenoid prosthesis 14 to the scapula S.

FIG. 5C illustrates stem 18 having a generally tubular cross-section with a plurality of bores 48 extending through sidewall 50 thereof. When stem 18 is inserted within canal C, adhesive 52 can be forced under pressure into the tubular stem 18 and out bores 48 to engage canal C when adhesive 52 cures. FIG. 5D illustrates stem 18 having a generally circular cross-section with a plurality of depressions 54 thereon. The canal C can be filled with adhesive when the stem 18 is inserted therein. When cured, the cured adhesive 52 within the depressions can secure stem 18 in canal C. Alternately stem 18 can be fabricated of a porous material and/or constructed to have a porous outer surface such that adhesive 52 can infiltrate the pores to secure stem 18 when cured. Other alternate embodiments can be contemplated, including, but not limited to various combinations of the embodiments illustrated in FIGS. 3, 4A-4C and 5A-5D. For example, the keel 232 illustrated in FIG. 4B can be used in combination with the fluted stem 18 illustrated in FIG. 5A.

To provide a satisfactory prosthetic joint, the surface of glenoid prosthesis 14 accommodating the head (or prosthetic ball) of the humerus should be relatively smooth. Where opening 28 is provided in front face 24, a plug of suitable material, e.g., bone cement, metal, or other suitable materials such as plastic, can be provided in opening 28 to maintain a smooth surface on front face 24. A detent or shoulder can be provided in opening 28 such that a corresponding shoulder or detent on the plug can secure it in place within the opening. Alternately, a wearing surface 56 can be secured to the front face 24 of the shell 16, as illustrated in FIG. 3. Wearing surface 56 can be secured by fastening means 58 and/or other securing means known for use in arthroplasty procedures, including adhesives, screws, pins, and the like. Alternately, glenoid prosthesis 14 can be fabricated, e.g., by casting, as a single unitary construct to correspond with the modular embodiments illustrated herein, thus eliminating the need for an opening therethrough.

Imaging techniques, including Computerized Axial Tomography/Computed Tomography (CAT/CT) scans, Magnetic Resonance Imaging (MRI), and other known imaging techniques, can be used to determine the dimensions of glenoid prosthesis 14 for use in shoulder arthroplasty for a particular patient. Images or scans of the shoulder area and scapula S can be used to determine the length and cross-sectional dimensions of the canal C, dimensions of the glenoid and relationships between the longitudinal axis of the canal C and the plane of the glenoid, including the angle between the axis and the plane and the location of their intersection. Based on the foregoing dimensions and relationships, glenoid prosthesis 14 can be dimensioned to fit securely against the prepared scapula S, including proscribing a length, diameter and taper of the stem 18, the dimensions of the shell 16 and the location and angle of stem 18 with respect to shell 16. Exemplary dimensions for the diameter of the stem 18 can range from about 2 mm to about 10 mm. Generally, the length of stem 18 can be less than about 200 mm.

Many additional changes in the details, materials, and arrangement of parts, herein described and illustrated, can be made by those skilled in the art. Accordingly, it will be understood that the following claims are not to be limited to the embodiments disclosed herein, can include practices otherwise than specifically described, and are to be interpreted as broadly as allowed under the law.

We claim:

1. A glenoid prosthesis for use in shoulder arthroplasty, comprising:
a shell component having a first face configured to contact a surface of a scapula, and
a stem configured to extend from the first face into a canal in a lateral border of the scapula and configured to engage within the canal for anchoring the glenoid prosthesis to the scapula.

2. The glenoid prosthesis of claim 1, wherein the shell has an opening therethrough, the stem being inserted into the canal through the opening, the glenoid prosthesis further comprising an attachment means for securing the shell to the stem.

3. The glenoid prosthesis of claim 2, wherein the stem is tapered to conform with a narrowing of the canal, and wherein the opening is tapered to conform with the stem, a dimension of the opening at the first face being smaller than a corresponding dimension of an end of the stem nearest the shell, the attachment means comprising wedging the stem within the opening.

4. The glenoid prosthesis of claim 2, wherein the attachment means comprises threads on an end of the stem nearest the shell and matching threads within the opening, wherein the stem is threaded into the opening to secure the shell to the stem.

5. The glenoid prosthesis of claim 2, wherein the attachment means comprises:
a flange on an end of the stem nearest the shell, and
a shoulder formed within the opening to accommodate the flange, wherein a force is exerted by the flange against the shoulder to secure the shell to the stem.

6. The glenoid prosthesis of claim 2, wherein the attachment means comprises:
a shoulder formed within the opening, and
a bolt having a threaded end engaging an end of the stem nearest the shell, and having an opposite flange end to engage the shoulder as the bolt is threaded on the stem.

7. The glenoid prosthesis of claim 1, wherein the stem is threaded for engaging the stem within the canal.

8. The glenoid prosthesis of claim 1, wherein the stem is fluted for engaging the stem within the canal.

9. The glenoid prosthesis of claim 1, wherein the stem has a longitudinal opening extending at least partway therethrough and a plurality of bores extending from an outer surface of the stem generally radially to intersect the longitudinal opening, the longitudinal opening and bores for receiving adhesive therethrough for engaging the stem within the canal.

10. The glenoid prosthesis of claim 1, wherein an outer surface of the stem comprises a plurality of barbs extending outwardly from the outer surface for engaging the stem within the canal.

11. The glenoid prosthesis of claim 1, wherein an outer surface of the stem comprises a plurality of depression in the outer surface for engaging with an adhesive within the canal.

12. The glenoid prosthesis of claim 1, wherein the stem comprises a porous outer surface for engaging with an adhesive within the canal, the porous outer surface configured to allow the adhesive to infiltrate therein.

13. The glenoid component of claim 1, wherein a length and diameter of the stem correspond to a length and diameter of the canal.

14. The glenoid component of claim 1, wherein an angle formed between the stem and the shell corresponds to an angle between the canal and a natural glenoid of the shoulder.

15. A method of performing shoulder arthroplasty, comprising:
preparing a glenoid region of a scapula to obtain access to a canal in a lateral border of the scapula,
preparing the glenoid region to accommodate a shell component of a glenoid prosthesis configured for articulation of a humeral head of the shoulder therein, and providing a stem configured to extend from the glenoid region into the canal and configured to engage within the canal for anchoring the glenoid prosthesis to the scapula.
